SEQUENCE LISTING (63 sequences)

SEQ ID NO: 1 - protein, 9 aa, Hepatitis C virus
CVNGVCWTV

SEQ ID NO: 2 - protein, 10 aa, Hepatitis C virus
KLVALGINAV

SEQ ID NO: 3 - protein, 9 aa, Hepatitis C virus
ALYDVVTKL

SEQ ID NO: 4 - protein, 9 aa, Human immunodeficiency virus
AVDLSHFLK

SEQ ID NO: 5 - protein, 10 aa, Hepatitis B virus
FLPSDFFPSV

SEQ ID NO: 6 - protein, 10 aa, Human immunodeficiency virus
QVPLRPMTYK

SEQ ID NO: 7 - protein, 11 aa, Human immunodeficiency virus
RLRDLLLIVTR

SEQ ID NO: 8 - protein, 11 aa, Hepatitis C virus
STLPETTTVRR

SEQ ID NO: 9 - protein, 10 aa, Human immunodeficiency virus
KRWIILGLNK

SEQ ID NO: 10 - protein, 9 aa, Human immunodeficiency virus
TPYDINQML

SEQ ID NO: 11 - protein, 9 aa, Human immunodeficiency virus
ILKEPVHGV

SEQ ID NO: 12 - protein, 8 aa, Human T-cell lymphotropic virus
LFGYPVYV

SEQ ID NO: 13 - protein, 9 aa, Influenza A virus
GILGFVFTL

SEQ ID NO: 14 - protein, 8 aa, Human immunodeficiency virus
YLKDQQLL

SEQ ID NO: 15 - protein, 9 aa, Epstein-Barr Virus
FLRGRAYGI

SEQ ID NO: 16 - protein, 9 aa, Human immunodeficiency virus
GEIYKRWII

SEQ ID NO: 17 - protein, 10 aa, Human cytomegalovirus
IAGNSAYEYV

SEQ ID NO: 18 - protein, 9 aa, Human immunodeficiency virus
DCKTILKAL

SEQ ID NO: 19 - protein, 9 aa, Human immunodeficiency virus
YLKDQQLYL

SEQ ID NO: 20 - protein, 9 aa, Human immunodeficiency virus
GGKKKYKLK

SEQ ID NO: 21 - protein, 9 aa, Hepatitis B virus
LLAQFTSAI

SEQ ID NO: 22 - protein, 10 aa, Hepatitis B virus
LLVPFVQWFV

SEQ ID NO: 23 - protein, 9 aa, Hepatitis B virus
WLSLLVPFV

SEQ ID NO: 24 - protein, 10 aa, Hepatitis C virus
YLLPRRGPRL

SEQ ID NO: 25 - protein, 9 aa, Hepatitis C virus
DLMGYIPLV

SEQ ID NO: 26 - protein, 10 aa, Hepatitis C virus
GVAGALVAFK

SEQ ID NO: 27 - protein, 9 aa, Hepatitis B virus
FLLAQFTSA

SEQ ID NO: 28 - protein, 9 aa, Hepatitis B virus
FLLSLGIHL

SEQ ID NO: 29 - protein, 9 aa, Hepatitis B virus
ALMPLYACI

SEQ ID NO: 30 - protein, 10 aa, Hepatitis C virus
LLFNILGGWV

SEQ ID NO: 31 - protein, 9 aa, Hepatitis C virus
FLLLADARV

SEQ ID NO: 32 - protein, 10 aa, Hepatitis C virus
LLALLSCLTV

SEQ ID NO: 33 - protein, 9 aa, Human papillomavirus
LLMGTLGIV

SEQ ID NO: 34 - protein, 9 aa, Human papillomavirus
YMLDLQPET

SEQ ID NO: 35 - protein, 9 aa, Human papillomavirus
FAFRDLCIV

SEQ ID NO: 36 - protein, 10 aa, Hepatitis B virus
ILLLCLIFLL

SEQ ID NO: 37 - protein, 9 aa, Hepatitis B virus
KLHLYSHPI

SEQ ID NO: 38 - protein, 9 aa, Hepatitis B virus
VLLDYQGML

SEQ ID NO: 39 - protein, 10 aa, Hepatitis B virus
LLPIFFCLWV

SEQ ID NO: 40 - protein, 9 aa, Hepatitis B virus
VLQAGFFLL

SEQ ID NO: 41 - protein, 9 aa, Human papillomavirus
TLGIVCPIC

SEQ ID NO: 42 - protein, 9 aa, Human papillomavirus
TLHEYMLDL

SEQ ID NO: 43 - protein, 9 aa, Human papillomavirus
GTLGIVCPI

SEQ ID NO: 44 - protein, 9 aa, Human immunodeficiency virus
VLAEAMSQV

SEQ ID NO: 45 - protein, 10 aa, Human immunodeficiency virus
LLWKGEGAVV

SEQ ID NO: 46 - protein, 9 aa, Human immunodeficiency virus
LLWKGEGAV

SEQ ID NO: 47 - protein, 9 aa, Human immunodeficiency virus
ILKEPVHGV

SEQ ID NO: 48 - protein, 9 aa, Human immunodeficiency virus
IVGAETFYV

SEQ ID NO: 49 - protein, 9 aa, Human papillomavirus
MLDLQPETT

SEQ ID NO: 50 - protein, 10 aa, Human papillomavirus
TIHDIILECV

SEQ ID NO: 51 - protein, 8 aa, Hepatitis C virus
STNPKPQK

SEQ ID NO: 52 - protein, 9 aa, Hepatitis C virus
GPRLGVRAT

SEQ ID NO: 53 - protein, 20 aa, Hepatitis C virus
YPWPLYGNEGLGWAGWLLSP

SEQ ID NO: 54 - protein, 10 aa, Hepatitis B virus
YLHTLWKAGV

SEQ ID NO: 55 - protein, 9 aa, Hepatitis B virus
PLLPIFFCL

SEQ ID NO: 56 - protein, 10 aa, Hepatitis B virus
ILSTLPETTV

SEQ ID NO: 57 - protein, 9 aa, Human immunodeficiency virus
IIGAETFYV

SEQ ID NO: 58 - protein, 9 aa, Human immunodeficiency virus
LWVTVYYGV

SEQ ID NO: 59 - protein, 9 aa, Human immunodeficiency virus
LMVTVYYGV

SEQ ID NO: 60 - protein, 10 aa, Hepatitis B virus
YLHTLWKAGI

SEQ ID NO: 61 - protein, 9 aa, Human immunodeficiency virus
DPKVKQWPL

SEQ ID NO: 62 - protein, 9 aa, Hepatitis B virus
WLSLLVPFV

SEQ ID NO: 63 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYAFYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRQAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

(19) United States
(12) Patent Application Publication
(10) Pub. No.: US 2009/0305408 A1
(43) Pub. Date: Dec. 10, 2009

(54) Method for the rapid expansion of antigen specific t-cell

(75) Inventor: Kyong-Mi Chang, Bryn Mawr, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Trustees of the University of Pennsylvania, Philadelphia, PA (US)

(21) Appl. No.: 11/920,069

(22) PCT Filed: May 10, 2006
(86) PCT No.: PCT/US06/17921

§ 371 Date: Dec. 8, 2008

Related U.S. Application Data
(60) Provisional application No. 60/679,795, filed on May 11, 2005.

(51) Int. Cl. C12N 5/08

(52) U.S. Cl. 435/372.3

(57) ABSTRACT

The present invention encompasses a method for expanding an antigen specific T cell from a population of cells. The method of the present invention comprises contacting a population of cells with an MHC restricted antigenic peptide, a cytokine and a co-stimulatory signal. The invention also encompasses compositions and kits comprising an antigen specific T cell.

### BACKGROUND OF THE INVENTION

Infectious viral diseases present a difficult public health problem for the world. There are few anti-viral pharmaceuticals, and vaccines almost exclusively provide prophylactic assistance, but offer no assistance once an infection has commenced. Examples of infectious viruses include viruses belonging to the following families: picornavirus, adenovirus, retrovirus, paramyxovirus, bunyavirus, papovavirus, herpesvirus, reovirus, poxvirus, togavirus, filovirus, parvovirus, calicivirus, hepadnavirus, orthomyxovirus, arenavirus, filovirus, rhabdovirus, coronavirus, and flavivirus.

Hepatitis C is a disease characterized by an inflammation of the liver caused by infection with the hepatitis C virus (HCV), a flavivirus. Numerous risk factors for becoming infected with HCV have been identified, including receiving a blood transfusion prior to July 1992; receiving blood, blood products, or solid organs from a donor who has hepatitis C; injecting illicit drugs or sharing needles with someone who has HCV; long term kidney dialysis; frequent workplace contact with blood; having sex with multiple partners or partners infected with HCV; sharing personal items, such as toothbrushes and razors, with someone who is infected with HCV; or birth to an HCV infected mother.

It is estimated that approximately 4 million people in the United States are infected with HCV, or about 1 in 70 to 1 in 100 people. HCV is often asymptomatic, and is first detected during a blood test for a routine physical or another medical procedures. If the infection has been present for many years, the liver may be permanently scarred, clinically known as cirrhosis. In many cases, there are no symptoms of the disease until cirrhosis has developed.

When symptoms of HCV infection are present, they can include jaundice, abdominal pain, fatigue, loss of appetite, nausea and vomiting, low grade fever, pale or clay-colored stools, dark urine, general itching, ascites and dilated veins in the esophagus.

There is presently no cure for HCV. The most common treatment includes administering interferon alpha or a combination of interferon alpha and ribavirin. Interferon alpha is administered by injection just under the skin and has a number of side effects, including flu-like symptoms, headaches, fever, fatigue, loss of appetite, nausea, vomiting, depression, and thinning hair. Treatment with interferon alpha may also interfere with the production of white blood cells and platelets. Ribavirin is a capsule taken twice daily, and the major side effect is severe anemia (low red blood cells). Ribavirin also causes birth defects.

HCV is one of the most common causes of chronic liver disease in the U.S. today. At least 80% of patients with acute hepatitis C ultimately develop chronic liver infection, and 20% to 30% develop cirrhosis. Between 1% and 5% of patients may develop liver cancer. Hepatitis C is now the number 1 cause for liver transplantation in the U.S.

Adoptive transfer is a term coined by Medawar (1954, Proc. Royal Soc. 143:58-80) to study allograft rejection. The term adoptive immunotherapy denotes the transfer of immunocompetent cells for the treatment of cancer or infectious diseases, including HCV (June, C. H., ed., 2001, In: Cancer Chemotherapy and Biotherapy: Principles and Practice, Lippincott Williams & Wilkins, Baltimore; Vonderheide et al., 2003, Immun. Research 27:1-15). Adoptive therapy can be considered as a strategy aimed at replacing, repairing, or enhancing the biological function of a damaged tissue or system by means of autologous or allogeneic cells.

Adoptive imumunotherapy has been used in the clinic to treat various viral infections. The first successful infusion of ex vivo expanded, polyclonal CD4 T cells that enabled a high degree of engraftment upon infusion, was performed using magnetic beads coated with anti-CD3 and anti-CD28 beads (αCD3/28 coated beads) to ex vivo expand T cells from HIV infected individuals (Levine et al., 2002, Nature Med. 8:47-53).

Coated beads comprising αCD3/28 deliver the signals needed for T cell activation and growth, render T cells resistant to infection by downregulating CCR5 and upregulating the expression of various ligands, the β-chemokines RANTES, Macrophage Inflammatory Protein-1 alpha (MIP-1α) and MIP-1β (Levine et al., 1996, Science 272:1939-1943; Riley et al., 1997, J. Immunol. 158:5545-5553 Carroll et al., 1997, Science 276: 273-276). Several phase I and II trials have demonstrated that infusing up to 3×10¹⁰ autologous CD4 T cells expanded using αCD3/28 coated beads into infected individuals is both safe and feasible (Carroll et al., 1997, Science 276:273-276; Levine et al., 2002, Nature Med. 8:47-53; Walker et al., 2000, Blood 96:467-474; Ranga et al., 1998, Proc. Natl. Acad. Sci. U.S.A 95:1201-1206). More importantly, sustained increases in the total lymphocyte count, the CD4 to CD8 T cell ratio, the fraction of cytokine-secreting T cells, and the ability to respond to recall antigens were observed, suggesting that adoptive T cell immunotherapy has the potential to restore at least limited immune function back to infected individuals (Levine et al., 2002, Nature Med. 8:47-53).

Given the gravity of viral infections in general and HCV infections in particular, and the promise of adoptive immunotherapy as a feasible means for treating viral infections, methods and compositions are needed to create specifically targeted lymphocytes for use in examining the immune response to viral infections and for treating viral diseases. The present invention meets this need.

### BRIEF SUMMARY OF THE INVENTION

The present invention includes a method of expanding a virus specific T cell in a population of cells comprising isolating said population of cells from a human, contacting said population of cells with an MHC restricted viral antigenic peptide, a cytokine and a co-stimulatory signal, wherein said co-stimulatory signal is an anti-CD3/anti-CD28 coated bead, thereby expanding a virus specific T cell from said population of cells.

In one aspect of the invention, the human was previously infected with said virus.

In another aspect of the invention, the T cell is specific for hepatitis C virus.

In yet another aspect of the invention, the cytokine is interleukin-2.

The present invention includes a method of enriching a population of cells for a virus specific T cell, the method comprising isolating said population of cells from a human, contacting said population of cells with an MHC restricted viral antigenic peptide, a cytokine and a co-stimulatory signal, wherein said co-stimulatory signal is an anti-CD3/anti-CD28 coated bead, thereby enriching a population of cells for a virus specific T cell.

In one aspect of the invention, the human was previously infected with said virus.

In another aspect of the invention, the T cell is specific for hepatitis C virus.

In yet another aspect of the invention, the cytokine is interleukin-2.

The present invention includes a method of inducing proliferation of a virus specific T cell, the method comprising contacting said cell with an MHC restricted viral antigenic peptide, a cytokine and a co-stimulatory signal, wherein said co-stimulatory signal is an anti-CD3/anti-CD28 coated bead, thereby inducing proliferation of a virus specific T cell.

In one aspect of the invention, the T cell is specific for hepatitis C virus.

In another aspect of the invention, the cytokine is interleukin-2.

The present invention includes a virus specific T cell generated by isolating a population of cells from a human, contacting said population of cells with an MHC restricted viral antigenic peptide, a cytokine and a co-stimulatory signal, wherein said co-stimulatory signal is an anti-CD3/anti-CD28 coated bead.

In one aspect of the invention, the T cell is specific for hepatitis C virus.

In another aspect of the invention, the cytokine is interleukin-2.

The present invention includes a kit for expanding a virus specific T cell from a population of cells, said kit comprising interleukin-2, an anti-CD3/anti-CD28 coated bead and an viral antigenic peptide, wherein said peptide is selected from the group consisting of the peptide set forth in SEQ ID NO:1, SEQ ID NO:2, SEQ ID NO:3, a HCV core protein 15-mer and a HCV NS3 15-mer.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph depicting the in vitro expansion of isolated peripheral blood mononuclear cells (PBMC) via simultaneous expansion with αCD3/28 beads at the ratios indicated, as well as IL-2 and HCV peptides.

FIG. 2 is a graph depicting the percentage of CD8 cells positive for HCV tetramers per total lymphocytes sampled after stimulation with αCD3/28 beads, IL-2 and HCV peptides. The asterisk indicates extra stimulation on Day 10 with 15-mer pools.

FIG. 3 is a series of images depicting CD8 T cells positive for HCV specific tetramers determined by FACS analysis of PBMC before and after stimulation with aCD3/28 beads, HCV peptides and IL-2. The number in each circle in each FACS plot indicates the percent of HCV tetramer positive CD8 cells/lymphoid cells for three HLA-A2 restricted HCV CTL epitopes at Days 0, 7 and 17.

FIG. 4 is the amino acid sequence of an HCV polypeptide from including the core and NS3 proteins, from which the 15-mers used in the present invention were derived (SEQ ID NO:63).

### DETAILED DESCRIPTION OF THE INVENTION

The invention relates to the discovery of methods for rapidly expanding virus specific T cells from a lymphocyte cell population. That is, as demonstrated by the data disclosed herein, the present invention includes a method of expanding viral specific lymphocytes, preferably CD8 cells, and enriching a population of lymphocytes for viral specific lymphocytes, preferably CD8 cells. The present invention further comprises lymphocytes expanded by the methods of the present invention.

### DEFINITIONS

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, “amino acids” are represented by the full name thereof, by the three-letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

By the term “applicator,” as the term is used herein, is meant any device including, but not limited to, a hypodermic syringe, a pipette, and the like, for administering the compounds and compositions of the invention.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

By the term “effective amount”, as used herein, is meant an amount that when administered to a mammal, causes a detectable level of T cell response compared to the T cell response detected in the absence of the compound. T cell response can be readily assessed by a plethora of art-recognized methods.

The skilled artisan would understand that the amount of the compound or composition administered herein varies and can be readily determined based on a number of factors such as the disease or condition being treated, the age and health and physical condition of the mammal being treated, the severity of the disease, the particular compound being administered, and the like.

“Instructional material,” as that term is used herein, includes a publication, a recording, a diagram, or any other medium of expression which can be used to communicate the usefulness of the composition and/or compound of the invention in the kit for effecting alleviating or treating the various diseases or disorders recited herein. Optionally, or alternately, the instructional material may describe one or more methods of alleviating the diseases or disorders in a cell or a tissue or a mammal, including as disclosed elsewhere herein.

The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively.

As used herein, the term “pharmaceutically acceptable carrier” means a chemical composition with which the active ingredient may be combined and which, following the combination, can be used to administer the active ingredient to a subject.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, to “treat” means reducing the frequency with which symptoms of a disease (i.e., viral infection, tumor growth and/or metastasis, or other effect mediated by decreased numbers and/or decreased activity of T cells, and the like) are experienced by a patient.

A “therapeutic” treatment is a treatment administered to a patient who exhibits signs of pathology for the purpose of diminishing or eliminating those signs and/or decreasing or diminishing the frequency, duration and intensity of the signs.

An “effective amount” of a compound is that amount of a cell (e.g., a T cell stimulated and/or expanded according to the present invention) which is sufficient to provide a detectable effect to a population of T cells, or to a mammal, to which the T cell is administered and/or contacted.

By the term “specifically binds,” as used herein, is meant an antibody which recognizes and binds with a cognate binding partner (e.g., a stimulatory and/or costimulatory molecule present on a T cell) protein present in a sample, but which antibody or ligand does not substantially recognize or bind other molecules in the sample.

### DESCRIPTION

The present invention relates to the surprising discovery that large amounts of antigen specific T cells can be expanded from human lymphocytes and human lymphocyte populations using an antigenic peptide, a cytokine and a co-stimulatory signal. Specifically, the present invention includes the discovery that viral specific T cells can be expanded and enriched from a population of peripheral blood mononuclear cells (PBMC) by contacting the population of PBMC with a cytokine, an antigenic peptide and a bead that provides a co-stimulatory signal.

### I. Methods

The invention encompasses a method to expand antigen specific T cells from a population of human lymphocytes. The compositions and methods of the present invention are particularly preferred for targeting host cells infected by viruses. CTL responses are an important component of the immune responses of most mammals to a wide variety of viruses, and the present invention provides a means to effectively stimulate a CTL response to virus-infected cells and treat or prevent such an infection in a host mammal. Thus the compositions and methods of the present invention are applicable to any virus presenting protein and/or peptide antigens. Such viruses include but are not limited to the following, pathogenic viruses such as influenza A and B viruses (FLU-A, FLU-B), human immunodeficiency type I and II viruses (HIV-I, HIV-II), Epstein-Barr virus (EBV), human T lymphotropic (or T-cell leukemia) virus type I and type II (HTLV-I, HTLV-II), human papillomaviruses types 1 to 18 (HPV-1 to HPV-18), rubella (RV), varicella-zoster (VZV), hepatitis B (HBV), hepatitis C (HCV), adenoviruses (AV), and herpes simplex viruses (HV). In addition, cytomegalovirus (CMV), poliovirus, respiratory syncytial (RSV), rhinovirus, rabies, mumps, rotavirus and measles viruses.

The method comprises isolating lymphocytes, preferably a primary peripheral blood mononuclear cell (PBMC), from a human donor, contacting the PBMC with an antigenic peptide, a cytokine and a co-stimulatory signal. In one embodiment of the present invention, the human donor has a prior history of infection with a virus. In one embodiment of the invention, the virus is HCV, HIV, influenza, hepatitis B virus, hepatitis A virus, hepatitis D virus, adenovirus, a flavivirus, cytomegalovirus, Epstein-Barr virus, herpes simplex virus 1, herpes simplex virus 2, varicella-zoster virus, human herpesvirus 6, papilloma virus, parvovirus B19, polyomavirus BK, polyomavirus JC, measles virus, rubella virus, human T-cell virus I and human T-cell virus II. Preferably the virus is HCV.

The method of the present invention further comprises contacting a PBMC with an antigenic peptide. The antigenic peptide used in the present invention is preferably a peptide that is a known antigen of a virus. That is, the antigenic peptide of the present invention is preferably an antigen that is expressed by a cell in the context of an MHC molecule when the cell is infected with a virus. In one embodiment, the antigen is an HCV NS3 1073 peptide, an HCV NS3 1406 peptide, an HCV NS5 2594 peptide, a 15-mer from the HCV core protein and a 15-mer from an HCV NS3 protein. The present invention further comprises additional MHC restricted CTL epitopes from other viruses, such as those disclosed herein. Even more preferably, the antigenic peptides/CTL epitopes used in the method of the present invention is restricted to HLA-A2.

Methods for identifying an antigenic peptide, in particular an MHC restricted viral antigenic peptide, are well known in the art and are disclosed in for example, U.S. Pat. Nos. 5,780,036, 5,783,567 and U.S. Pat. No. 6,419,931, all of which are incorporated by reference in their entirety herein. MHC class I restricted CTL recognize processed viral peptides that are presented in an antigen binding site on the class I molecule formed between two alpha helices, with the floor of the groove formed by a beta sheet. A number of methods have been used to determine the identity of the presented peptides. The most precise definition has come from elution of such peptides from class I molecules, revealing consistent motifs shared by peptides presented by the same class I molecule. The binding motifs are characterized by anchor residues which serve as contact sites between the peptide and specific pockets in the class I binding groove. The amino and carboxy termini of epitopic peptides fit into the A and F pockets of this groove, respectively, due to hydrogen bonding. The amino terminal anchors of the peptides are variable in position and number, whereas the carboxy anchor is always at the C-terminus, with the side chain pointing directly into the bottom of the F pocket. Although there is considerable heterogeneity among amino terminal anchor residues, the F pocket appears to place more restrictions on the amino acids it will accommodate, such that either leucine, isoleucine, arginine, tyrosine, valine or phenylalanine is at the C-terminus of over 95% of known epitopes. Motif predictions for antigenic peptides have now been generated for a large number of HLA class I molecules, and are known in the art. Such MHC class I viral peptides include, but are not limited to the peptides listed in the table below.

| | | SEQ |
|---|---|---|
| Virus | Peptide | ID NO: |
| HIV nef84-94 | AVDLSHFLK | 4 |
| HBc18-27 | FLPSDFFPSV | 5 |
| HIV nef73-82 | QVPLRPMTYK | 6 |
| HIV-1 NL43 env | RLRDLLLIVTR | 7 |
| gp41 768-778 | | |
| HCV 141-151 | STLPETTTVRR | 8 |
| HIV gag p24 265-274 | KRWIILGLNK | 9 |
| HIV-2 | TPYDINQML | 10 |
| HIV RT | ILKEPVHGV | 11 |
| HTLV-1, Tox 12-19 | LFGYPVYV | 12 |
| Influenza A, | GILGFVFTL | 13 |
| M1 58-66 | | |
| HIV gp41 586-593 | YLKDQQLL | 14 |
| EBV EBNA-3 | FLRGRAYGI | 15 |
| HIV gag261-269 | GEIYKRWII | 16 |
| HCMV, gB 619-628 | IAGNSAYEYV | 17 |
| HIV gag331-339 | DCKTILKAL | 18 |
| HIV gp41 586-593 | YLKDQQLYL | 19 |
| HIV | GGKKKYKLK | 20 |
| HBV POL 1117 | LLAQFTSAI | 21 |
| HBV ENV 338 | LLVPFVQWFV | 22 |
| HBV ENV 335 | WLSLLVPFV | 23 |
| HCV | YLLPRRGPRL | 24 |
| HCV | DLMGYIPLV | 25 |
| HCV | GVAGALVAFK | 26 |
| HBV ENV 1116 | FLLAQFTSA | 27 |
| HBV POL 1147 | FLLSLGIHL | 28 |
| HBV POL 1245 | ALMPLYACI | 29 |
| HCV | LLFNILGGWV | 30 |
| HCV | FLLLADARV | 31 |
| HCV | LLALLSCLTV | 32 |
| HPV16 E7 | LLMGTLGIV | 33 |
| HPV16 E7 | YMLDLQPET | 34 |
| HPV16 E6 | FAFRDLCIV | 35 |
| HBV ENV | ILLLCLIFLL | 36 |
| HBV POL | KLHLYSHPI | 37 |
| HBV ENV | VLLDYQGML | 38 |
| HBV ENV | LLPIFFCLWV | 39 |
| HBV ENV | VLQAGFFLL | 40 |
| HPV16 E7 | TLGIVCPIC | 41 |
| HPV16 E7 | TLHEYMLDL | 42 |
| HPV16 E7 | GTLGIVCPI | 43 |
| HIV | VLAEAMSQV | 44 |
| HIV | LLWKGEGAVV | 45 |
| HIV | LLWKGEGAV | 46 |
| HIV | ILKEPVHGV | 47 |
| HIV | IVGAETFYV | 48 |
| HPV16 E7 | MLDLQPETT | 49 |
| HPV16 E6 | TIHDIILECV | 50 |
| HCV | STNPKPQK | 51 |
| HCV | GPRLGVRAT | 52 |
| HCV | YPWPLYGNEGLG | 53 |
| | WAGWLLSP | |
| HBV POL | YLHTLWKAGV | 54 |
| HBV ENV | PLLPIFFCL | 55 |
| HBV NUC (CORE) | ILSTLPETTV | 56 |
| HIV | IIGAETFYV | 57 |
| HIV | LWVTVYYGV | 58 |
| HIV | LMVTVYYGV | 59 |
| HBV POL | YLHTLWKAGI | 60 |
| HIV pol185-193 | DPKVKQWPL | 61 |
| HBVadr-ENV | WLSLLVPFV | 62 |
| (S Ag335-343) | | |

The present invention also comprises contacting a PBMC with a cytokine. Preferably, the cytokine is a T-cell growth factor cytokine, such as IL-2, IL-15, IFN γ and the like. Even more preferably, the cytokine is IL-2. Recombinant IL-2 (rIL2) is available commercially from, for example, Sigma (St. Louis, Mo.).

The invention disclosed herein further comprises contacting a PBMC with a co-stimulatory signal. Preferably, the co-stimulatory signal is a bead comprising antibodies that specifically bind to a T cell and provide a co-stimulatory signal. Examples of co-stimulatory signals include, but are not limited to anti-CD3 antibody and anti-CD28 antibody. Methods for making anti-CD3/anti-CD28 antibody coated beads (αCD3/28) are known in the art and are described in, for example, Levine et al. (2002, Nature Med. 8:47-53; Levine et al., 1996, Science 272:1939-1943).

As demonstrated by the data disclosed herein, the PBMC are contacted with αCD3/28 beads at different ratios of beads to cells. Preferably the PBMC are contacted at a ratio of about 1:1 (αCD3/28:cell), even more preferably at a ratio of about 1:2, still more preferably at a ratio of about 1:3, even more preferably at a ratio of about 1:4, still more preferably at a ratio of 1:5. The skilled artisan, when equipped with the present disclosure and the data disclosed herein, can determine other αCD3/28:cell ratios, including ratios, for example, of 1:10, 1:20, 1:50 and 1:100.

As disclosed elsewhere herein, the PBMC is contacted with a antigenic peptide, a cytokine and a co-stimulatory signal as a substantially simultaneous occurrence. That is, the antigenic peptide, cytokine and co-stimulatory signal are provided to the cell at about the same time, or in close temporal proximity to each other. The present invention further encompasses contacting a PBMC with an antigenic peptide, a cytokine and a co-stimulatory signal at different times. That is, the present invention encompasses performing the methods disclosed herein as distinctly separate events spaced by a longer period of time.

The methods of the present invention expand the T cell, preferably a CD8 T cell, specifically in that only the T cells expanded are specific for the virus from which the antigenic peptide was derived. Thus, where the T cell to be expanded is present in a mixture of cells, the T cells of interest will be induced to proliferate and expand in cell number. Further, as demonstrated by the data disclosed herein, a large number of T cells that are specific for a viral antigen are expanded and the mixture of cells is enriched for a viral specific T cells. In addition, the T cell can be further purified using a wide variety of cell separation and purification techniques, such as those known in the art and/or described elsewhere herein.

As would be appreciated by the skilled artisan, based upon the disclosure provided herein, the T cell of interest need not be identified or isolated prior to expansion using the methods of the present invention. This is because the methods of the present invention are selective for the type of antigenic peptide from any given virus and will expand the T cell(s) responsive to the antigenic peptide.

The present invention also includes a method for specifically expanding a T cell population subset. More particularly, the method comprises contacting a population of T cells comprising at least one T cell of a subset of interest with an antigenic peptide, a cytokine and a co-stimulatory signal capable of expanding that T cell. This is because, as demonstrated by the data herein, the methods of the present invention induces proliferation of the T cell, thereby specifically expanding a T cell population subset. One skilled in the art would understand, based upon the disclosure provided herein, that T cell subsets include T helper (T_{H1} and T_{H2}) CD4 expressing, cytotoxic T lymphocyte (CTL) (Tc1 or Tc2) T regulatory (T_{REG}), T_{c/s}, naïve, memory, central memory, effector memory, and γδT cells. Therefore, cell populations enriched for a particular T cell subset can be readily produced using the method of the invention.

The invention further encompasses a method for inducing a T cell response to an antigen, preferably an viral antigen in a mammal. The method comprises isolating a PBMC from an animal, contacting a PBMC with an antigenic peptide, preferably an MHC restricted antigenic peptide, a cytokine and a co-stimulatory signal. Preferably the cytokine is IL-2 and the co-stimulatory signal is an aCD3/38 bead. Once sufficient numbers of antigen-specific T cells are obtained using the methods of the present invention to expand the T cell, the antigen-specific T cells so obtained are administered to the mammal, thereby inducing a T cell response to the antigen in the mammal. This is because the data disclosed herein amply demonstrate that antigen-specific T cells can be readily produced by stimulating resting T cells using the methods of the invention.

### II. Compositions

The present invention encompasses an isolated T cell produced by the methods of the present invention. Preferably, the T cell is a CD8 cell. Even more preferably, the CD8 cell is specific for a viral antigen. The skilled artisan would appreciate, based upon the disclosure provided herein, that numerous viral antigens can be used to produce an almost limitless variety of virus specific T cells. That is, there is extensive knowledge in the art regarding the antigenic peptides that are expressed in the context of an MHC molecule on an infected cell. Thus, one of skill in the art, using the methods disclosed herein, could produce a T cell specific for any virus.

The T cell produced by the methods of the present invention can be used in the preparation and use of a pharmaceutical compositions comprising a virus specific T cell of the invention as an active ingredient. Such a pharmaceutical composition may consist of the active ingredient alone, as a combination of at least one active ingredient (e.g., an effective dose of an virus specific T cell) in a form suitable for administration to a subject, or the pharmaceutical composition may comprise the active ingredient and one or more pharmaceutically acceptable carriers, one or more additional (active and/or inactive) ingredients, or some combination of these.

As used herein, the term “pharmaceutically acceptable carrier” means a chemical composition with which the active ingredient may be combined and which, following the combination, can be used to administer the active ingredient to a subject.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

In addition to the active ingredient, a pharmaceutical composition of the invention may further comprise one or more additional pharmaceutically active agents. Particularly contemplated additional agents include anti-viral agents such as protease inhibitors, nucleoside analogs, reverse transcriptase inhibitors, interferon alpha, ribavarin, and the like.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The virus specific T cell of the invention and/or T cells expanded using the methods of the present invention, can be administered to an animal, preferably a human. When the T cells expanded using the methods of the invention are administered, the amount of cells administered can range from about 1 million cells to about 300 billion. While the precise dosage administered will vary depending upon any number of factors, including but not limited to, the type of animal and type of disease state being treated, the age of the animal and the route of administration.

The virus specific T cell can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

A virus specific T cell may be co-administered with the various other compounds (cytokines, chemotherapeutic and/or antiviral drugs, among many others). Alternatively, the compound(s) may be administered an hour, a day, a week, a month, or even more, in advance of a virus specific T cell, or any permutation thereof. Further, the compound(s) may be administered an hour, a day, a week, or even more, after administration of a virus specific T cell, or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds and the virus specific T cell, and the like.

### III. Kits

The invention includes various kits which comprise the various reagents for producing the virus-specific T cells of the present invention. Such reagents include, but are not limited to, an antigenic peptide, a cytokine and a co-stimulatory signal. In one embodiment of the kit of the present invention, the antigenic peptide is from a virus, including HCV, HIV, influenza, hepatitis B virus, hepatitis A virus, hepatitis D virus, adenovirus, a flavivirus, cytomegalovirus, Epstein-Barr virus, herpes simplex virus 1, herpes simplex virus 2, varicella-zoster virus, human herpesvirus 6, papilloma virus, parvovirus B19, polyomavirus BK, polyomavirus JC, measles virus, rubella virus, human T-cell virus I and human T-cell virus II. In one embodiment of the invention, the cytokine is IL-2 and the co-stimulatory signal is an αCD3/28. The kit of the present invention further comprises an applicator, and an instructional material which describes use of the kit to perform the methods of the invention. Although exemplary kits are described below, the contents of other useful kits will be apparent to the skilled artisan in light of the present disclosure. Each of these kits is included within the invention.

The invention includes a kit for treating a viral infection in a human. That is, the present invention includes an MHC restricted antigenic peptide, a cytokine and a co-stimulatory signal for inducing proliferation of a T cell that is then administered to a human or other mammal in order to treat a viral infection. This is because the methods of the present invention induce proliferation of a T cell that is specific for a viral antigen and the T cell can be used to treat a viral infection. The kit is used pursuant to the methods disclosed in the invention. Briefly, the kit may be used to administer a virus specific T cell of the invention to a mammal. This is because, as more fully disclosed elsewhere herein, the data disclosed herein demonstrate that contacting a T cell with an MHC restricted antigenic peptide, a cytokine and a co-stimulatory signal mediates proliferation, specificity and enrichment of a T cell from a population of cells. The T cells produced using this kit can be administered to an animal to achieve therapeutic results.

The kit further comprises an applicator useful for administering a T cell expanded by the methods of the present invention. The particular applicator included in the kit will depend on, e.g., the method used to administer the T cell. Such applicators are well-known in the art and may include, among other things, a pipette, a syringe, a dropper, and the like. Moreover, the kit comprises an instructional material for the use of the kit. These instructions simply embody the disclosure provided herein.

The kit can further include a pharmaceutically-acceptable carrier. The composition is provided in an appropriate amount as set forth elsewhere herein. Further, the route of administration and the frequency of administration are as previously set forth elsewhere herein.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

### Rapid Expansion of Antigen-Specific T-Cells from Lymphocytes

Peripheral blood mononuclear cells (PBMC) were isolated from an individual that spontaneously recovered from hepatitis C virus infection. The isolated PBMC were stimulated starting on Day 0 according to the protocols in Table 1.

TABLE 1

| | | | aCD3/28 |
|---|---|---|---|
| | | | Bead to |
| Protocol | HCV CTL Epitopes | rIL-2 | Cell Ratio |
| 1:1 + A2 + IL2 | HLA-A2 Restricted NS3 1073, | 100 U/ml | 1:1 |
| | NS3 1406 and NS5 2594 at 10 | | |
| | μg/ml each | | |
| 1:5 + A2 + IL2 | HLA-A2 Restricted NS3 1073, | 100 U/ml | 1:5 |
| | NS3 1406 and NS5 2594 at 10 | | |
| | μg/ml each | | |
| 1:1 + 15-mer | Pool of overlapping HCV-core | 100 U/ml | 1:1 |
| pool + IL2 | and NS3 15-mers at 1 μM for | | |
| | each peptide | | |
| 1:1 + 15-mer | Pool of overlapping HCV-core | 100 U/ml | 1:5 |
| pool + IL2 | and NS3 15-mers at 1 μM for | | |
| | each peptide | | |

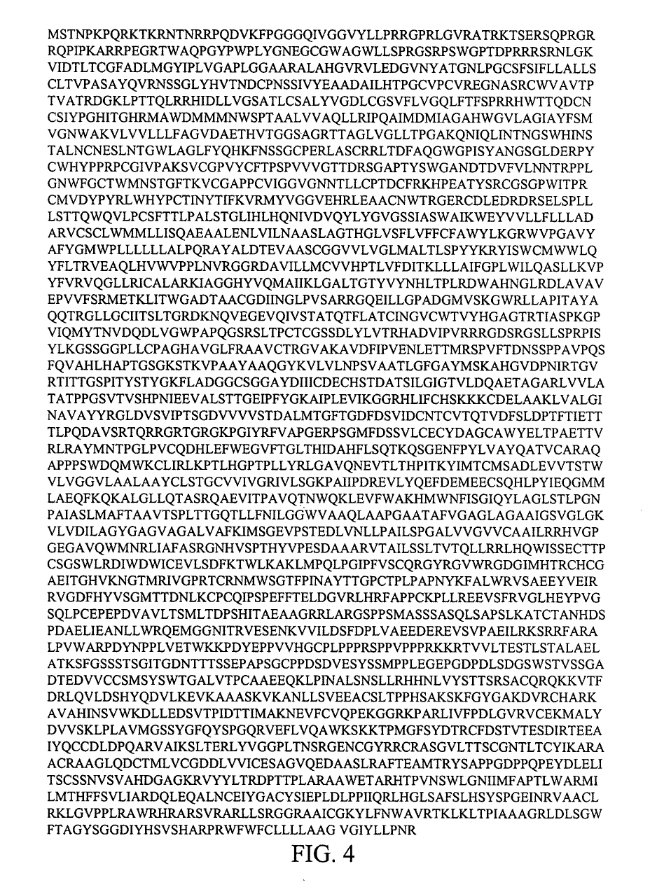

The sequences or references for the Hepatitis C Virus Cytotoxic T-Lymphocyte (HCV CTL) epitopes are as follows: NS3 1073: CVNGVCWTV (SEQ ID NO:1), NS3 1406: KLVALGINAV (SEQ ID NO:2), NS5 2594: ALYDVVTKL (SEQ ID NO:3). The amino acid sequence of the HCV genome, including the core protein and the NS3 protein are set forth in FIG. 4 (SEQ ID NO:63). Construction of the pool of overlapping HCV-derived 15-mer peptides from HCV core and NS3 protein are described in, for example, Anthony et al. (2004, J. Immunology, 172: 4907-4916). Anti-CD3/anti-CD28 monoclonal antibody coated beads (αCD3/28 beads) are generated as described in, for example, Levine et al. (2002, Nature Med. 8:47-53; Levine et al., 1996, Science 272:1939-1943).

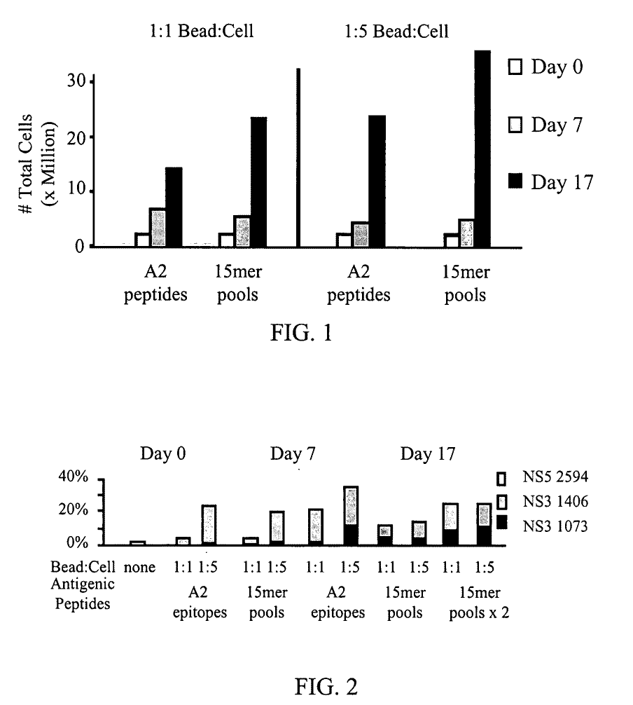

Following the initial stimulation, the cultures were maintained with rIL2 (100 U/ml) in complete media every 3-4 days and examined at day 7 and day 17. In some cases, the cultures were split on day 10 into two separate wells of which one well received only rIL2 while the other well was stimulated with a second dose of the same antigenic peptides as on day 0. This resulted in 10-15 fold increase in overall cell number (FIG. 1).

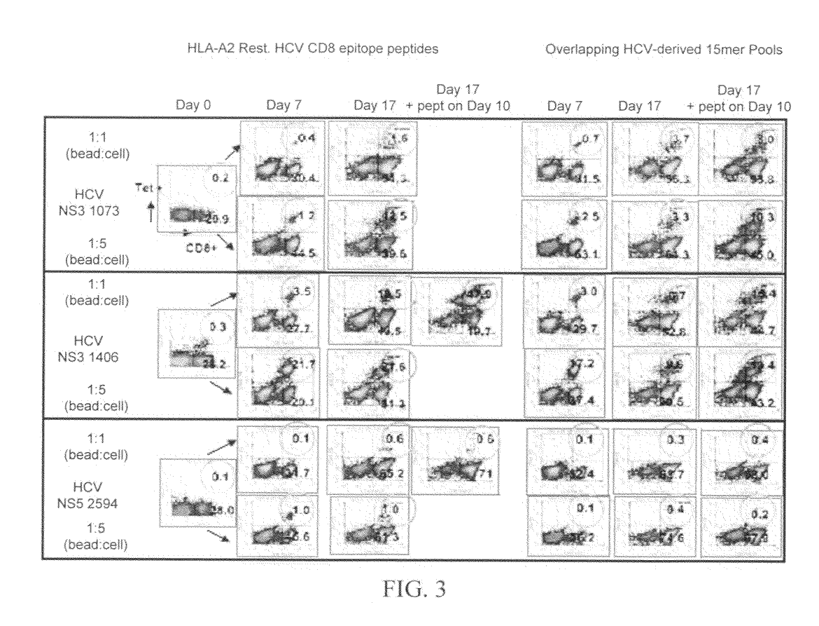

The expansion protocol presently described resulted in a marked enrichment of HCV-specific T cells when compared to the low frequency of detectable HCV-specific T cells at day 0 (before stimulation). The enrichment of HCV-specific T cells was detected using MHC/peptide tetramers specific for HLA-A2 restricted CD8 CTL epitopes and by intracellular cytokine staining (FIGS. 2 and 3).

As demonstrated by the data disclosed herein, simultaneous stimulation with antigens and aCD3/28 beads with rIL2 is an efficient method to rapidly expand antigen-specific T cells in vitro.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of expanding a virus specific T cell in a population of cells, said method comprising isolating said population of cells from a human, contacting said population of cells with an MHC restricted viral antigenic peptide, a cytokine and a co-stimulatory signal, wherein said co-stimulatory signal is an anti-CD3/anti-CD28 coated bead, thereby expanding a virus specific T cell from said population of cells.

2. The method of claim 1, wherein said human was previously infected with said virus.

3. The method of claim 1, wherein said T cell is specific for hepatitis C virus.

4. The method of claim 1, wherein said cytokine is interleukin-2.

5. A method of enriching a population of cells for a virus specific T cell, the method comprising isolating said population of cells from a human, contacting said population of cells with an MHC restricted viral antigenic peptide, a cytokine and a co-stimulatory signal, wherein said co-stimulatory signal is an anti-CD3/anti-CD28 coated bead, thereby enriching a population of cells for a virus specific T cell.

6. The method of claim 5, wherein said human was previously infected with said virus.

7. The method of claim 5, wherein said T cell is specific for hepatitis C virus.

8. The method of claim 5, wherein said cytokine is interleukin-2.

9. A method of inducing proliferation of a virus specific T cell, the method comprising contacting said cell with an MHC restricted viral antigenic peptide, a cytokine and a co-stimulatory signal, wherein said co-stimulatory signal is an anti-CD3/anti-CD28 coated bead, thereby inducing proliferation of a virus specific T cell.

10. The method of claim 10, wherein said T cell is specific for hepatitis C virus.

11. The method of claim 10, wherein said cytokine is interleukin-2.

12. A virus specific T cell generated by isolating a population of cells from a human, contacting said population of cells with an MHC restricted viral antigenic peptide, a cytokine and a co-stimulatory signal, wherein said co-stimulatory signal is an anti-CD3/anti-CD28 coated bead.

13. The method of claim 13, wherein said T cell is specific for hepatitis C virus.

14. The method of claim 13, wherein said cytokine is interleukin-2.

15. A kit for expanding a virus specific T cell from a population of cells, said kit comprising interleukin-2, an anti-CD3/anti-CD28 coated bead and an viral antigenic peptide, wherein said peptide is selected from the group consisting of the peptide set forth in SEQ ID NO:1, SEQ ID NO:2, SEQ ID NO:3, a HCV core protein 15-mer and a HCV NS3 15-mer.
